(19) United States
(12) Patent Application Publication
(10) Pub. No.: US 2009/0186111 A1
(43) Pub. Date: Jul. 23, 2009

(54) Novel Bacteria and Pharmaceutically Active Products Obtained Therefrom

(76) Inventors: Zeev Trainin, Tel Aviv (IL); Gabriel Leitner, Mazkeret Batia (IL); Eugenia Lubashevsky, Bneiy Aish (IL); Sofia Svaransky, Lod (IL)

Correspondence Address: MOORE & VAN ALLEN PLLC, P.O. BOX 13706, Research Triangle Park, NC, 27709, US

(21) Appl. No.: 11/658,686

(22) PCT Filed: Jul. 24, 2005
(86) PCT No.: PCT/IL2005/000785

§ 371 Date: Jan. 26, 2007

Related U.S. Application Data
(60) Provisional application No. 60/590,939, filed on Jul. 26, 2004.

(51) Int. Cl. A61K 35/74; A61P 31/12; A61P 17/02

(52) U.S. Cl. 424/780; 435/252.1

(57) ABSTRACT

A substance obtainable from Staphylococcus Aureus bacteria or from a Staphylococcus Aureus bacteria culture, the substance characterized in having at least one of the following characteristics: (a) stimulates proliferation of bovine T-lymphocytes in vitro; (b) accelerates healing of skin wounds in mice and pigs; (c) prevents AIDS-like disease (ALD) in mice infected with ALD virus (ALD-V) when injected substantially simultaneously with the virus; and (d) reduces the viral load in Rhesus Macaques infected with the hybrid simian/human immunodeficiency virus (SHIV) when injected into chronically infected Macaques, or a derivative or fraction of said substance which retains at least one of the characteristics of the substance. Also disclosed is a novel Staphylococcus Aureus bacteria, and a pharmaceutical composition comprising as active ingredient the substance and a pharmaceutically acceptable excipient.

### FIELD OF THE INVENTION

This invention relates to a novel Staphylococcus Aureus bacteria and pharmaceutically active products which may be obtained therefrom.

### BACKGROUND OF THE INVENTION

The international pandemic of HIV infection and AIDS is expanding rapidly. Since the early 1980s more than 40 million individuals have contracted HIV, and almost 12 million have consequently died. In 1997 alone nearly 6 million people (approximately 16,000 cases a day) acquired HIV and some 2.3 million perished from it, including 460,000 children. Today, aggressive treatment can preserve health and prolong life for those HIV-positive patients who have access to optimal medical care. However, treatments are imperfect and demanding. Thus, better medications that are less expensive and easier to administer are needed.

There also exists an urgent need for medications effective in the healing of skin wounds and burns.

### SUMMARY OF THE INVENTION

A biological substance has now been discovered with potential antiviral and wound healing properties. The biological substance may be isolated from a novel Staphylococcus Aureus bacteria culture.

Thus, the present invention provides a substance obtainable from Staphylococcus Aureus bacteria or from a Staphylococcus Aureus bacteria culture, the substance characterized in having at least one of the following characteristics:
(a) stimulates proliferation of bovine T-lymphocytes in vitro;
(b) accelerates healing of skin wounds in mice and pigs;
(c) prevents AIDS-like disease (ALD) in mice infected with ALD virus (ALD-V) when injected substantially simultaneously with the virus; and
(d) reduces the viral load in Rhesus Macaques infected with the hybrid simian/human immunodeficiency virus (SHIV) when injected into chronically infected Macaques,

or a derivative or fraction of said substance which retains at least one of the characteristics of the substance.

In a preferred embodiment of the invention, the substance has at least two of characteristics (a), (b), (c) and (d). In a more preferred embodiment, the substance has at least three of characteristics (a), (b), (c) and (d). In a most preferred embodiment, the substance has all of characteristics (a), (b), (c) and (d). In another preferred embodiment, the substance is a supernatant of a Staphylococcus Aureus bacteria culture or a molecular entity obtainable therefrom. Still another preferred embodiment of the invention relates to a derivative, homologue or analog of the molecular entity.

The phrase “substantially simultaneously” in item (c) above comes to include injection within 0.5 hours of each other.

The terms “derivative, homologue or analog” include various chemical and molecular processing of the substance of the invention, wherein the resulting derivative retains at least characteristics (c) and (d) of the substance, more preferably characteristics (b), (c) and (d), and most preferably characteristics (a) to (d).

A biologically active fraction may be isolated from the substance of the invention which has a MW in the range of 7-13 kDa and which has at least characteristics (c) and (d) of the substance. Another biologically active fraction may be isolated from the substance of the invention which has a MW less than or equal to 3.5 kDa and which has at least characteristics (c) and (d) of the substance. Under certain conditions, as described below, the substance of the invention has been found to be heat-resistant.

In a most preferred embodiment of the invention, the Staphylococcus Aureus bacteria culture is prepared from a S. aureus bacterial species deposited at the National Collection of Agricultural and Industrial Microorganisms, Budapest, Hungary, on Jul. 6, 2004 under the accession number NCAIM (P) B 001321. This bacteria species is an aspect of the invention.

The substance of the invention has been surprisingly found to have therapeutic properties.

In a first aspect of the present invention, the substance may be used to treat mammalian wounds manifested, e.g. by diabetes, burns, trauma and subcutaneous trauma, various surgical procedures, and various forms of dermatitis.

In a preferred embodiment of this aspect of the invention, a wound-healing pharmaceutical composition may be formulated using the substance of the invention together with excipients and carriers to produce a mixture in gel, lotion, cream or ointment form. The inventive composition can be in powder form as well.

The present invention may be formulated as necessary with additives used commonly in the pharmaceutical sciences, such as surfactants, oils and fats, polyhydric alcohols, lower alcohols, thickening agents, UV absorbents, light scattering agents, preservatives, antioxidants, antibiotics, chelating agents, pH regulators, flavoring agents, pigments and water.

Examples of surfactants include polyoxyethylene (hereinafter abbreviated as POE-branched alkyl ethers such as POE-octyldodecyl alcohol and POE-2-decyltetradecyl alcohol, POE-alkyl ethers such as POE-oleyl alcohol ether and POE-cetyl alcohol ether, sorbitan esters such as sorbitan monooleate, sorbitan monoisostearate and sorbitan monolaurate, POE-sorbitan esters such as POE-sorbitan monooleate, POE-sorbitan monoisostearate and POE-sorbitan monolaurate, fatty acid esters of glycerol such as glyceryl monooleate, glyceryl monostearate and glyceryl monomyristate, POE-fatty acid esters of glycerol such as POE-glyceryl monooleate, POE-glyceryl monostearate and POE-glyceryl monomyristate, POE-dihydrocholesterol ester, POE-hardened castor oil, POE-hardened castor oil fatty acid esters such as POE-hardened castor oil isostearate, POE-alkylaryl ethers such as POE-octylphenol ether, glycerol esters such as glycerol monoisostearate and glycerol monomyristate, POE-glycerol ethers such as POE-glycerol monoisostearate and POE-glycerol monomyristate, polyglycerol fatty acid esters such as diglyceryl monostearate, decaglyceryl decastearate, decaglyceryl decaisostearate and diglyceryl diisostearate and other nonionic surfactants; potassium salts, sodium salts, diethanolamine salts, triethanolamine salts, amino acid salts and other salts of higher fatty acids such as myristic acid, stearic acid, palmitic acid, behenic acid, isostearic acid and oleic acid, the above alkali salts of ether carboxylic acids, salts of N-acylamino acids, N-acylsalconates, higher alkylsulfonates and other anionic surfactants; alkylamine salts, polyamine, aminoalcohol fatty acids, organic silicone resin, alkyl quaternary ammonium salts and other cationic surfactants; and lecithin, betaine derivatives and other amphoteric surfactants.

Examples of oils and fats include vegetable oils and fats such as castor-oil, olive oil, cacao oil, camellia oil, coconut oil, wood wax, jojoba oil, grape seed oil and avocado oil; animal oils and fats such as mink oil and egg yolk oil; waxes such as beeswax, whale wax, lanolin, carnauba wax and candelilla wax; hydrocarbons such as liquid paraffin, squalene, microcrystalline wax, ceresine wax, paraffin wax and vaseline; natural or synthetic fatty acids such as lauric acid, myristic acid, stearic acid, oleic acid, isostearic acid and behenic acid; natural or higher alcohols such as cetanol, stearyl alcohol, hexyldecanol, octyldecanol and lauryl alcohol; and esters such as isopropyl myristate, isopropyl palmitate, octyldodecyl myristate, octyldodecyl oleate and cholesterol oleate.

Examples of polyhydric alcohols include ethylene glycol, polyethylene glycol, propylene glycol, 1,3-butyrene glycol, 1,4-butyrene glycol, dipropylene glycol, glycerol, diglycerol, triglycerol, tetraglycerol and other polyglycerols, glucose, maltose, maltitose, sucrose, fructose, xylitose, sorbitol, maltotriose, threitol and erythritol.

Examples of thickening agents include naturally-occurring high molecular substances such as sodium alginate, xanthene gum, aluminum silicate, quince seed extract, gum tragacanth, starch, collagen and sodium hyaluronate; semi-synthetic high molecular substances such as methyl cellulose, hydroxyethyl cellulose, carboxymethyl cellulose, soluble starch and cationized cellulose; and synthetic high molecular substances such as carboxyvinyl polymer and polyvinyl alcohol.

Examples of UV absorbents include p-aminobenzoic acid, 2-ethoxyethyl p-methoxycinnamate, isopropyl p-methoxycinnamate, butylmethoxybenzoylmethane, glyceryl-mono-2-ethylhexanoyl-di-p-methoxybenzophenone, digalloyl trioleate, 2,2′-dihydroxy-4-methoxybenzophenone, ethyl-4-bishydroxypropylaminobenzoate, 2-ethylhexyl-2-cyano-3,3′-diphenyl acrylate, ethylhexyl p-methoxycinnamate, 2-ethylhexyl salicylate, glyceryl p-aminobenzoate, homomethyl salicylate, methyl o-aminobenzoate, 2-hydroxy-4-methoxybenzophenone, amyl p-dimethylaminobenzoate, 2-phenylbenzoimidazole-5-sulfonic acid and 2-hydroxy-4-methoxybenzophenone-5-sulfonic acid.

Examples of preservatives include benzoates, salicylates, sorbates, dehydroacetates, p-oxybenzoates, 2,4,4′-trichloro-2′-hydroxydiphenyl ether, 3,4,4′-trichlorocarbanilide, benzalkonium chloride, hinokitiol, resorcinol and ethanol.

Examples of antioxidants include tocopherol, ascorbic acid, butylhydroxyanisole, dibutylhydroxytoluene, nordihydroguairetic acid and propyl gallate.

Examples of chelating agents include sodium edetate and sodium citrate.

Examples of antibiotics include penicillin, neomycin, cephalothin, potassium permanganate, selenium sulfide, erythromycin, bacitracin, tetracyclin, chloramphenicol, vancomycin, nitrofurantoin, acrisorcin, chlorodontoin, and flucytosine.

Some of these additives function to enhance the efficacy of the composition by increasing the stability or percutaneous absorbability of the essential components of the present invention.

Also, any dosage form is acceptable, whether in solution, emulsion, powder dispersion, or others. Applicability is wide, including fundamental dosage forms such as lotions, emulsions, creams and gels.

In addition to those stated above, suitable vehicles, carriers and adjuvants include water, vaseline, petrolatum, mineral oil, vegetable oil, animal oil, organic and inorganic waxes, polymers such as xanthanes, gelatin, cellulose, collagen, starch, kaolin, carrageenan, gum arabic, synthetic polymers, alcohols, polyols, and the like. The carrier can also include sustained release carrier such as lypizomes, microsponges, microspheres, or microcapsules, aqueous base ointments, water in oil or oil in water emulsions, gels or the like.

The invention also includes a method for healing a wound of a subject comprising administrating to the subject the substance of the invention.

The invention further comprises use of the substance of the invention in the preparation of a pharmaceutical composition.

The dose administered to an animal, particularly a human, in the context of the present invention should be sufficient to effect a therapeutic response over a reasonable time frame. The dose will be determined by the strength of the particular compositions employed and the condition of the person. The size of the dose and the frequency of application also will be determined by the existence, nature, and extent of any adverse side effects that may accompany the administration of a particular composition.

The pharmaceutical composition of the present invention may be employed to treat diabetic ulcers, healing resistant wounds, bed sores, burns, trauma wounds, subcutaneous trauma and various forms of dermatitis.

In a second aspect of the present invention, the substance of the invention may be used in the prevention or treatment of infection by the human immunodeficiency virus (HIV) as well as by animal retroviruses, and the treatment of consequent pathological conditions such as AIDS. The term “HIV” in this specification includes HIV and related animal retroviruses. Treating AIDS or preventing or treating infection by HIV is defined as including, but not limited to, treating a wide range of states of HIV infection: AIDS, ARC (AIDS related complex), both symptomatic and asymptomatic, and actual or potential exposure to HIV. For example, the substance of this invention may be useful in treating infection by HIV after suspected past exposure to HIV by, e.g., blood transfusion, organ transplant, exchange of body fluids, bites, accidental needle stick, or exposure to patient blood during surgery.

For these purposes, the substance of the present invention may be administered orally, parenterally (including subcutaneous injections, intravenous, intramuscular, intrasternal injection or infusion techniques), by inhalation spray, or rectally, in dosage unit formulations containing conventional non-toxic pharmaceutically-acceptable carriers, adjuvants and vehicles.

Thus, in accordance with the present invention there is further provided a method of treating and a pharmaceutical composition for treating HIV infection and AIDS. The treatment involves administering to a patient in need of such treatment a pharmaceutical composition comprising a pharmaceutical carrier and a therapeutically effective amount of the substance of the present invention, or a pharmaceutically acceptable salt thereof.

These pharmaceutical compositions may be in the form of orally-administrable suspensions or tablets; nasal sprays; sterile injectable preparations, for example, as sterile injectable aqueous or oleagenous suspensions or suppositories.

When administered orally as a suspension, these compositions are prepared according to techniques well-known in the art of pharmaceutical formulation and may contain microcrystalline cellulose for imparting bulk, alginic acid or sodium alginate as a suspending agent, methylcellulose as a viscosity enhancer, and sweetners/flavoring agents known in the art. As immediate release tablets, these compositions may contain microcrystalline cellulose, dicalcium phosphate, starch, magnesium stearate and lactose and/or other excipients, binders, extenders, disintegrants, diluents and lubricants known in the art.

When administered by nasal aerosol or inhalation, these compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

The injectable solutions or suspensions may be formulated according to known art, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid.

When rectally administered in the form of suppositories, these compositions may be prepared by mixing the drag with a suitable non-irritating excipient, such as cocoa butter, synthetic glyceride esters or polyethylene glycols, which are solid at ordinary temperatures, but liquidify and/or dissolve in the rectal cavity to release the drug.

Dosage levels of the order of 0.001 to 5.0 or 10.0 grams-per-day are useful in the treatment or prevention of the above-indicated conditions, with oral doses two-to-five times higher. For example, infection by HIV may be effectively treated by the administration of from 1.0 to 50 milligrams of the compound per kilogram of body weight from one to four times per day. In one preferred regimen, dosages of 100-400 mg every six hours are administered orally to each patient. It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drag combination, the severity of the particular condition, and the host undergoing therapy.

The substance of this invention may be administered orally to humans in a dosage range of 0.01 to 1000 mg/kg body weight in divided doses. One preferred dosage range is 0.1 to 200 mg/kg body weight orally in divided doses. Another preferred dosage range is 0.5 to 100 mg/kg body weight orally in divided doses. For oral administration, the compositions are preferably provided in the form of tablets containing 1 to 1000 milligrams of the active ingredient, particularly 1, 5, 10, 15, 20, 25, 50, 75, 100, 150, 200, 250, 300, 400, 500, 600, 750, 800, 900, and 1000 milligrams of the active ingredient for the symptomatic adjustment of the dosage to the patient to be treated. It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The present invention is also directed to combinations of the substance of the invention with one or more agents useful in the treatment of AIDS. For example, the substance of this invention may be effectively administered, whether at periods of pre-exposure and/or post-exposure, in combination with effective amounts of the AIDS antivirals, immunomodulators, anti-infectives, or vaccines known to those of ordinary skill in the art.

A therapeutically effective amount of the substance of the present invention may be useful in the inhibition of HIV protease, or in “salvage” therapy; i.e., the substance can be used to treat HIV infection, AIDS, or ARC in HIV-positive subjects whose viral load achieved undetectable levels via conventional therapies employing known protease inhibitors, and then rebounded due to the emergence of HIV mutants resistant to the known inhibitors.

As used herein, the term “composition” is intended to encompass a product comprising the specified ingredients in the specified amounts, as well as any product which results, directly or indirectly, from combination of the specified ingredients in the specified amounts.

The expression “pharmaceutically acceptable” means that the carrier, diluent or excipient must be compatible with the other ingredients of the formulation and not deleterious to the recipient thereof.

The term “subject,” (alternatively referred to herein as “patient”) as used herein refers to an animal, preferably a mammal, most preferably a human, who has been the object of treatment, observation or experiment.

The term “therapeutically effective amount” as used herein means that amount of active compound or pharmaceutical agent that elicits the biological or medicinal response in a tissue, system, animal or human that is being sought by a researcher, veterinarian, medical doctor or other clinician, which includes alleviation of the symptoms of the disease being treated.

Another aspect of the invention relates to a method for preparing a supernatant according to the invention, the method comprising:
(a) growing a Staphylococcus Aureus bacteria culture in a bacterial growth medium from a bacteria species equivalent to that deposited at the National Collection of Agricultural and Industrial Microorganisms, Budapest, Hungary, on Jul. 6, 2004 under the accession number NCAIM (P) B 001321; and
(b) separating the bacteria and concentrating the supernatant.

The method may further comprise the steps of:
(c) applying the substance of step (b) to an RP-18 column; and
(d) eluting an active fraction from the column, said fraction having at least one of the characteristics (a), (b), (c) and (d) of the substance of the invention.

Although the embodiment of the invention described below relates to a bacterial supernatant, the invention also relates to derivatives, fractions and molecular entities which may be isolated from the supernatant, and which have at least one of the activities which characterize the substance of the invention. The skilled man of the art will know how to extract and purify such derivatives, fractions and molecular entities from the supernatant. For example, the supernatant may be applied to a chromatography column, filtered or undergo electrophoresis as is well known in the art. The activity of various fractions may be ascertained using the assays described below, and those fractions having the determined activity pooled and concentrated. These standard chemical and biochemical methods are well known in the art and are described in detail in various textbooks and manuals.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 shows a Bradford pattern of the fractions obtained from a Sep-Pak cartridge;

FIG. 2 shows analytical separation of LSLT on a RP-18 column. 0.5 ml of LSLT was applied to a 10 ml RP-18 column which was developed at 1.5 ml/min collecting 1.5 ml/fraction;

FIG. 3 shows a preparative separation of LSLT on a RP-18 column. 40 ml of LSLT was applied to a 100 ml RP-18 column which was developed at 1.0 ml/min collecting 10 ml/fraction. Solid lines represent pooled fractions;

FIG. 4 shows chromatography on various matrices (Table III) of the 3 fractions of LSLT (FIG. 3);

FIG. 5 shows activity assays for the different fractions depicted in FIG. 3. Bars indicate proliferation assays; + indicates bioassays in mice;

FIG. 6 shows Viral RNA loads in SHIV89.6pd-infected rhesus macaques before, during and following treatment with LSLT (Phase 1). Arrows indicate times of treatment; and

FIGS. 7A & 7B shows viral RNA loads in rhesus macaques during and following treatment with LSLT (Group A; 7A) or saline (Group B; 7B)—Phase 2. Arrows indicate times of treatment.

### DETAILED DESCRIPTION OF AN EMBODIMENT OF THE INVENTION

### Example I

### Characterization of Novel Bacteria

A novel Staphylococcus Aureus bacteria was isolated from a bovine sub-clinical udder infection. The bacteria, named Staphylococcus aureus LSLT1111, was found to have the following properties:
Basic features: coagulase positive, non hemolytic.
Phage typing: untypable.
Microbiological test: 168 rRNA gene-800 bp fragment; forward 100%, reverse 100% (discriminate from Staphylococcus heamolyticus, multiplex PCR reaction was applied).
Biochemical and enzymatic characterization: see Table I
Antibiogram of S. aureus LSLT1111: see Table II

TABLE I
Biochemical and enzymatic characterization
of S. aureus LSLT1111

| | |
|---|---|
| GLU Glucose | + |
| FRU Fructose | + |
| MAL Maltose | + |
| LAC Lactose | + |
| TRE Trehalose | + |
| MAN Manitol | + |
| RAF Rafinose | + |
| RIB Ribose | + |
| CEL Celobiose | − |
| NIT Nitrates (reduction) | + |
| VP _Actoin Production) | + |
| β Gal β Galctosidase | − |
| ArgA Arginine Arylamidase | − |
| PAL Alkaline Phosphatase | + |
| PyrA Pyrrolidonyl Arylamidase | + |
| NOVO Novobiocin (Resistance) | − |
| SAC Sucrose | + |
| NAG N-Acetyl-glucosamine | + |
| TUR Turanose | + |
| ARA Arbinose | − |
| β GUR β Glucuronidase | − |
| URE Urease | − |
| ADH Arginin dihydrolase | + |
| ODC Ornithine decaboxilase | − |
| ESC Esculine (hydrolysis) | − |

The biochemical and enzyme characterization was performed by ID-32 API STAPH (Bio Merieux Vitex, Inc. Mo. USA).

TABLE II
Antibiogram of S. aureus LSLT1111

| | Diameter of | |
|---|---|---|
| | clear area (mm) | Sensitivity to antibiotic |
| Penicillin | 9.0 | Resistant |
| Ampicillin | 12.6 | Resistant |
| Meticillin | 23.3 | Sensitive |
| Amox. Clav. ac | 28.0 | Sensitive |
| Cephalothin | 28.8 | Sensitive |
| Neomycin | 14.7 | Resistant/Sensitive |
| Gentamicin | 16.6 | Sensitive |
| Tetracyclin | 19.8 | Sensitive |
| Chloramphenicol | 18.6 | Sensitive |
| Sulpha/trimethoprim | 18.8 | Sensitive |
| Erythromycin | 17.3 | Resistant/Sensitive |
| Lincomycin | 16.7 | Resistant/Sensitive |
| Quinolones | 17.0 | Resistant/Sensitive |
| Vancomycin | 11.9 | Sensitive |

The bacteria LSLT1111 was deposited in accordance with the Budapest Treaty at the National Collection of Agricultural and Industrial Microorganisms, Budapest, Hungary, on Jul. 6, 2004 under the accession number NCAIM (P) B 001321.

### Example II

### Production of Bacterial Supernatants of LSLT1111

The bacterium was grown in Columbia broth supplemented with 0.1% D-glucose, yeast extract and 0.5% NaCl (Difco, Detroit, Mich.) at 37° C. for 24 h and harvested by centrifugation at 3000×g for 15 min. at 4° C. The crude supernatant was collected, filtered through 0.2 μm pore-size membranes and was concentrated 1:10 (volume) in cellulose tubular membrane (Nominal MWCO: 3500, Cellu. Sep. Texas USA) by polyethylene glycol 35,000 (Fluka, Switzerland) at 4° C., dialyzed against PBS (pH 7.2, 4° C., 48 h).

The protein level in the concentrated supernatant solution was assayed with Bradford reagent (Bio-Rad, UK) and the solution was then stored at −20° C. This supernatant solution is one embodiment of the substance of the invention and was named LSLT.

### Example III

### Biochemical Characterization of LSLT and Preparation of Active Fraction

### Materials and Methods

Cell Proliferation assay—see Example V below.
Bioassay in mice—see Example VI below.

### 1. Size Determination by Ultrafiltration

The apparent molecular weight of the substance LSLT was analyzed by dialysis membranes with known cutoffs, using an equilibrium dialysis system. The ability to collect and analyze both the filtrate and the retentate allowed examination of the transport of the material across membranes of cutoffs: 3.5, 7 and 13 kDa. The various fractions were analyzed by the proliferation assay. The active fraction of the substance was found to be retained by the first two membranes but not by the 13 kDa membrane, where the active fraction could be collected from the filtrate fraction. These observations led to the conclusion that the molecular weight of the active fraction of the substance is between 7 and 13 kDa.

### 2. Hydrophobic Chromatography on RP-18

RP-18 is a silica-based solid matrix, bearing (CH₂)₁₈ hydrocarbon chains. It readily interacts with the hydrophobic moieties of peptides and proteins as well as hydrophobic materials. The more hydrophobic the compound, the higher is the concentration of ethanol required for its elution from the column. RP-18 is available as a 1 ml cartridge (“Sep-Pak”), for low scale solid phase extraction, or as a chromatographic column with higher capacity and resolution than the cartridge.

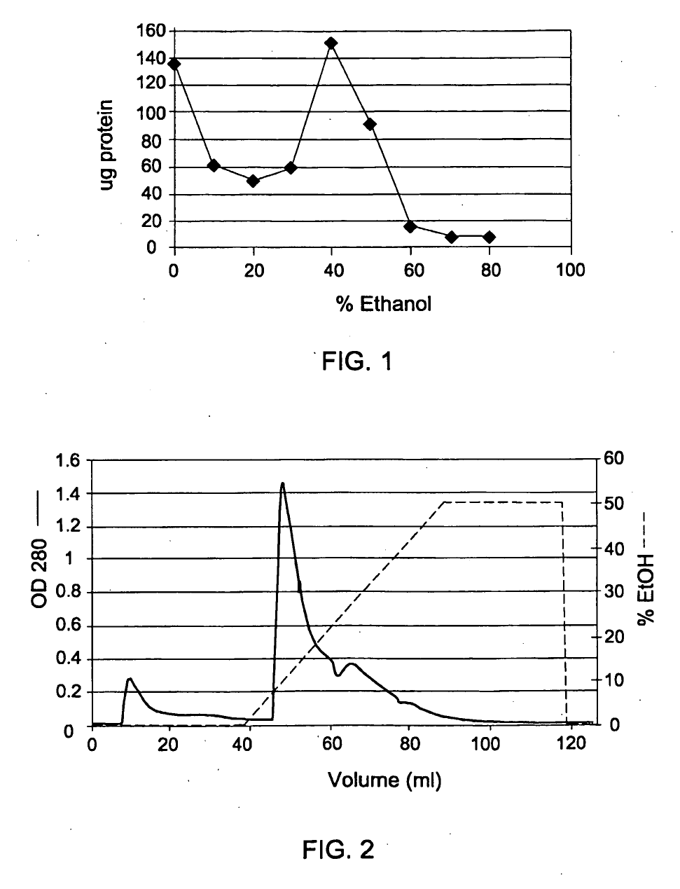

The isolation of the active fraction was first attempted on the RP-18 Sep-Pak cartridge. The cartridge was washed with water and 1 ml of LSLT was applied to it. The flow-through fraction was collected and the cartridge was further washed with water. Then the cartridge was washed with 2 ml fractions of 10, 20, 30, 40, 50, 60, 70, 80, 90 and 100% ethanol. These fractions were evaporated in a Speed-Vac apparatus and re-dissolved in 2 ml of water, each. FIG. 1 depicts the Bradford pattern of the fractions obtained with a peak at 40-50% ethanol.

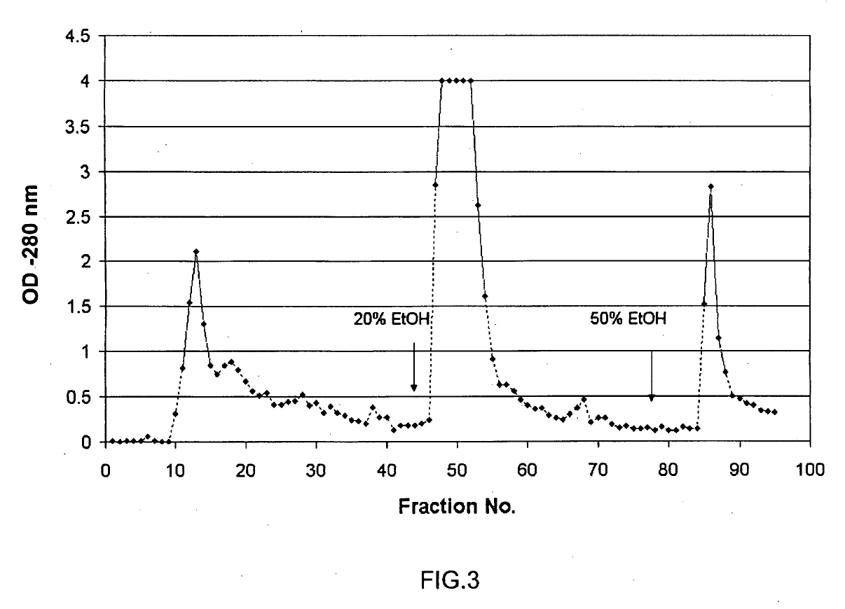

In the mouse bioassay and in the cell proliferation assay, activity was found to fractionate between the flow-through and 20% ethanol fractions. FIG. 2 depicts a typical run of the LSLT on an analytical RP-18 column. As shown in this figure, part of the material was eluted before introducing ethanol, while upon ethanol addition a major peak eluted within the 10-20% ethanol fractions followed by a minor peak at higher ethanol concentrations. Accordingly, a preparative system was set up to achieve processing of large LSLT preparations. The system contained a 100 ml RP-18 column onto which 40 ml fractions of LSLT were applied. The column was first washed with PBS until Fraction I was eluted, then with 20% ethanol in PBS (Fraction II) and finally with 50% Ethanol in PBS (Fraction III) (FIG. 3). The last two fractions were evaporated in a SpeedVac apparatus to remove the ethanol and then checked for proliferation activity and in the mouse bioassay. Similarly to previous results, activities were obtained in the first two fractions. The specific activities of these fractions were higher than that of the original material. The conclusion of these experiments was that the active fraction contains a highly hydrophilic component.

The RP-18 purification protocol may be applied for the large scale preparation of the LSLT active fraction.

### 3. Chromatography on Other Matrices

After characterizing the binding of LSLT onto RP-18, a relatively highly hydrophobic matrix, its binding to other, less hydrophobic, hydrophilic and ion exchange matrices was examined. These matrices appear in Table III, along with the elution conditions from each of them.

TABLE III
Matrices used in the chromatographic evaluation of LSLT

| | | | Application | First | Second | |
|---|---|---|---|---|---|---|
| No. | Group | Matrix | Buffer | Wash | Wash | Remarks |
| 1. | Strong | RP-18 | A | B | C | Hydrophobic |
| | Hydrophobic | | | | | Interactions |
| 2. | Weak | Phenyl | A | B | C | Aromatic |
| | Hydrophobic | | | | | Interactions |
| 3. | | Cyano | A | B | C | Aliphatic |
| | | | | | | Interactions |
| 4. | Hydrophilic | Diol | C | B | A | Hydrogen Bonds |
| 5. | | Silica | C | B | A | Polar Interactions |
| 6. | | Amino | C | B | A | Hydrogen Bonds |
| 7. | Ion Exchange | Anion | D | A | E | Negative Charges |
| 8. | | Cation | E | A | D | Positive Charges |

Buffers used: A - PBS, pH 7.4; B - 20% Ethanol in PBS; C - 50% Ethanol in PBS; D - 0.2M Borate Buffer, pH 9.0; E - 10% Acetic acid.

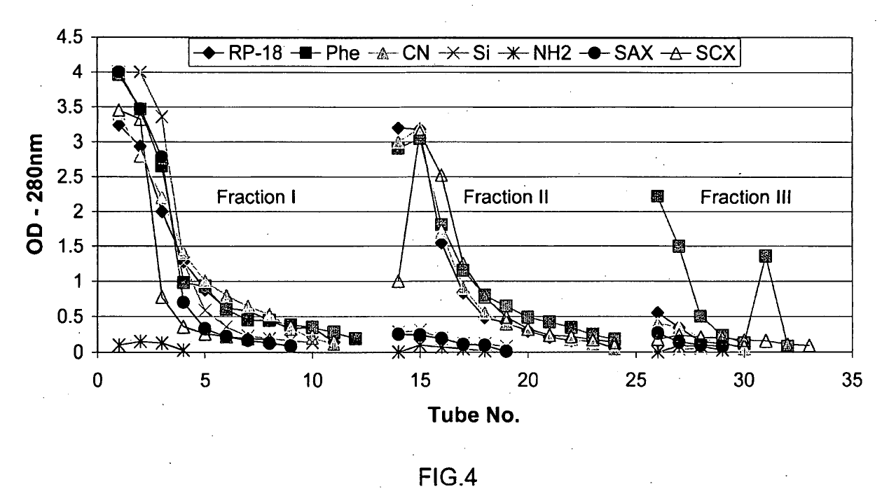

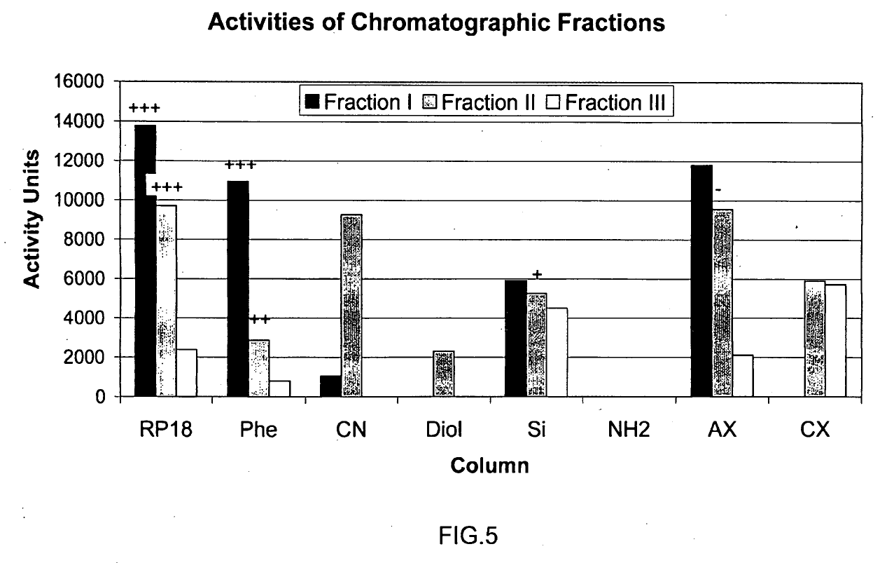

The weak hydrophobic columns (Phenyl-, CN) were developed under conditions similar to the RP-18 column, namely, loaded in PBS and eluted with increasing concentrations of ethanol. The hydrophilic columns (Diol-, Amino and silica) were developed in a reversed order, namely, loaded at high ethanol concentrations and eluted with a lower ethanol concentration and finally with PBS. The two ionic exchange columns, a positive and a negative one, which bind materials by charge interactions, were developed with appropriate pH gradients. The fractions obtained from these columns were subjected to the cell proliferation assay as well as the mouse bioassay. FIG. 4 and FIG. 5 depict the results (OD and activity) obtained from these columns.

FIGS. 4 and 5 summarize the OD patterns and the activities obtained with the various columns. As shown in FIG. 4, the hydrophobic column Phenyl bound LSLT similarly to the RP18 column and the active fraction was segregated between Fractions I and II. The less hydrophobic CN column yielded a similar binding curve but the biological activity (mainly in Fraction II) was much lower. With the hydrophilic column of silica practically none of the LSLT was bound to the column. However, Fraction II obtained from this column, which hardly contained any protein, showed a considerable activity by proliferation but very little activity in the mouse bioassay. With the amino column all the material was lost during the chromatography, probably representing non-reversible binding. No activity was detected in any of the fractions. The anion exchange column SAX, showed similar results to the silica column—very little material bound to the column but Fraction II yielded quite considerable activity in the proliferation assay but not in the mouse assay. In contrast, the cation exchange column SCX bound, and released, some active fraction of LSLT.

4. Conclusions from the Chromatographic Assays

The active fraction in LSLT seems to be slightly hydrophobic as it segregates between the unbound and the weakly bound fractions of the RP-18 and Phenyl columns. The hydrophilic column of silica showed interesting results. Although very little protein was eluted in Fraction II a considerable activity was detected in this fraction, indicating a high specific activity. Similar results were obtained with the anion exchange column. It is pertinent to note that there is a difference between the proliferation and the mouse assay results. For example, Fraction II of SAX has proliferation activity but no activity was detected in mice, while Fraction II from the phenyl column showed low proliferation activity and high activity in mice. In addition, the activity results did not always correlate with protein concentrations, e.g. Fractions II and III of the silica and SAX columns.

These results indicate the following properties for the active fraction from LSLT—(a) the compound is slightly hydrophobic but with a considerable amount of polar groups capable of forming hydrogen bonds (OH, NH₂, COOH); (b) it has negative charges at slightly basic pH values but a low amount of positive groups at low pH→Basic pK value.

### 5. Precipitation

During sample preparation for the Normal Phase columns it was observed that LSLT underwent precipitation in the presence of high concentrations of ethanol and acetonitrile.

### Example IV

### Healing of Skin Wounds in Mice

### Materials and Methods

Mice: mature Swiss-type
Infection: A virulent strain of Staphylococcus Aureus (1×10³ bacteria/ml liquid medium) was injected sc into the backs of the mice. The bacteria caused an infection and after 7 days, gangrene appeared in the skin. On the seventh day, the gangrenous skin was removed, the wound size was measured, and the mice underwent treatment. The wound was measured every 4-7 days until a scab was formed.

### Treatment:

Method #1: 4 drops (1 ml) of LSLT are dripped on the wound (4 trials);

Method #2: sc injection of 1 ml of LSLT (3 trials).

The treatments were carried out once a day for 3-4 consecutive days. The experimental design is summarized in Table IV. The treatments were carried out with crude LSLT and with fractions of the crude LSLT fractionated according to size, as follows: (1) between 5 and 100 kDa; (2) between 5 and 10 kDa. The control contained various unrelated proteins.

TABLE IV
Experimental design of wound treatment
with LSLT or LSLT fractions in mice.

| State¹ | | Experiments² | Num.³ | LSLT | 5-100 | 5-10 | CB | PBS |
|---|---|---|---|---|---|---|---|---|
| (1) | | 1 | 3 | 5 | — | — | — | 5 |
| | | 2 | 3 | 4 | — | — | — | 4 |
| | | 3 | 3 | 5 | 5 | 5 | 5 | 3 |
| | | 4 | 4 | 5 | 5 | 5 | 5 | 7 |
| (2) | 24 | 1 | 1 | 4 | — | — | — | 5 |
| | 24 | 2 | 1 | 12 | — | — | 12 | 12 |
| | 0 | | 1 | 12 | — | — | 12 | 12 |
| | 24 | 3 | 1 | 10 | — | 5 | 5 | — |

¹Treatment started on (1) day of skin removal; (2) at the time of inoculation or 24 h thereafter
²In set 2, time of treatments (hrs)
³Number of treatments
— Not done
CB placebo

### Results:

The results of two different experiments are summarized in Tables V and VI. From Table V it may be seen that LSLT as well as all 3 fractions caused a significant reduction in the wound size after 7 days, as opposed to 30% reduction in the control. On day 11 the wounds treated with LSLT (crude or fractions) were close to complete recovery while the control treatment showed a wound size of 30-90% of the original size. The difference between the treated and control mice is even more pronounced in the experiment summarized in Table VI, from which can be seen that already on day 3 the wound size of the treated mice is reduced by over 50% while in the control mice, the reduction is insignificant.

TABLE V
Dynamic of wound healing process in mice
treated with LSLT and its fractions.

| | Treatment² | | | | |
|---|---|---|---|---|---|
| Time¹ | LSLT | 10-100 | 5-10 | CB | PBS |
| 0 | 0 | 0 | 0 | 0 | 0 |
| 7 | 59.7 | 62.8 | 62.6 | 32.4 | 0 |
| 11 | 95.7 | 91.5 | 85.0 | 72.0 | 8.5 |
| 18 | 98.7 | 95.8 | 100 | 87.0 | 72.0 |
| 35 | 100 | 100 | 100 | 98.2 | 72.0 |

¹Time (days) after treatment
²Percentage of wound healing
CB placebo

TABLE VI
Dynamic of wound healing process in mice
treated with LSLT and its fractions.

| | Treatment² | | | | |
|---|---|---|---|---|---|
| Time¹ | LSLT | 10-100 | 5-10 | CB | PBS |
| 0 | 0 | 0 | 0 | 0 | 0 |
| 1 | 39.2 | 17.2 | 44.8 | 5.1 | 5.2 |
| 3 | 75.7 | 54.9 | 54.8 | 17.7 | 4.2 |
| 7 | 84.2 | 70.6 | 77.7 | 22.0 | 25.3 |
| 11 | 97.7 | 92.6 | 85.2 | 57.1 | 59.0 |
| 19 | 100 | 95.0 | 92.0 | 95.0 | 76.0 |
| 25 | 100 | 100 | 100 | 98.2 | 100 |

¹Time (days) after treatment
²Percentage of wound healing
CB placebo

### Example V

### Stimulation of Proliferation of Bovine T-Lymphocytes

### Materials & Methods

LSLT was tested at 3 dilutions: 1:102, 1:103 and 1:104.

Cells: bovine blood cells which were passed through a Ficoll-Hypaque gradient (Pharmacia, Upsala Sweden) by centrifugation at 600 g for 30 min. at RT. 0.1 ml of 1×10⁶ cells/ml RPMI-1640 medium containing 5% FCS, 100 u/ml penicillin and 100 u/ml streptomycin (Biological Products, Beit HaAmek, Israel) were inserted into microtiter plate wells (Nunc, Denmark). The plates were incubated under CO₂ for 72 hours at 37° C. A positive control comprised 5 μ/ml of Con A. 1 μCi of H³ thymidine (Amersham, U.K.) was added to each well 16-18 hours prior to harvesting. Harvesting was carried out using a Cell Harvester (Flow Lab., U.K.) and the counting was done using a Scintillation Analyzer TC 1600 (Packard, US). The results are expressed in cpm.

### Results

Representative results for a number of batches of LSLT are shown in Table VII. However, all of the batches which were assayed showed stimulation activity of between 30-70% of the positive control, and values of 8-30 times the background count. In other words, LSLT stimulates activity of lymphocytes, mainly T-lymphocytes as determined by a fluorescent antibody cell sorter (FACS) (Con A specifically activates T-lymphocytes).

TABLE VII
Proliferation activity of LSLT batches expressed in cpm

| LSLT | Cow | | | Dilutions | | |
|---|---|---|---|---|---|---|
| batch | No. | Background | Con A | 1:100 | 1:1000 | 1:10000 |
| 22 | 2365 | 1897 | 87801 | 20603 | 33152 | ND |
| 22 | 2368 | 3168 | 52318 | 17049 | 14950 | ND |
| 26 | 2314 | 1389 | 72375 | 23160 | 9720 | ND |
| 28 | 2314 | 1389 | 72375 | 36750 | 21495 | 18175 |
| 29 | 2314 | 1389 | 72375 | 34881 | 23733 | 16270 |
| 30 | 2314 | 1389 | 72375 | 36939 | 16292 | 11969 |
| 31 | 2427 | 3005 | 56936 | 44911 | 34736 | 21211 |
| 32 | 2427 | 3005 | 56936 | 42099 | 36836 | 26219 |
| 33 | 2427 | 3005 | 56936 | 34411 | 27786 | 22836 |

### Example VI

### Effect of LSLT on ALD in Mice

A mouse model of AIDS disease is described in Nature Medicine 3:37-41 (1971). The AIDS-like disease (ALD) develops in mice injected with ALD virus (ALD-V), resulting in an enlargement of the spleen (splenomegaly) beginning between day 10-14 after injection and peaking on day 18-21. Subsequently, there is a reduction in spleen size with a disappearance of symptoms for a period of several weeks.

Animals: Female Balb-C mice, 8 weeks old, 20-22 grams each.

Virus: ALD-V produced from plasma of infected mice. 0.2 ml per injection.

Treatment: injection of LSLT at a concentration of 400-600 mg protein/ml, 0.2 ml per unit, substantially simultaneously with the virus.

The mice were divided into four groups of 5 mice each:
I. injection of 0.2 ml PBS s/c (negative control)
II. injection of 0.2 ml LSLT s/c (test sample)
III. injection of 0.2 ml LSLT (known active fraction which acts as a positive control);

After 20 minutes, the mice of groups I, II and III are injected with 0.2 ml of virus;
IV. 3 mice received LSLT alone without virus (toxicity control).

On day 20, the mice were sacrificed and their spleens weighed. The body weight was also determined. A group calculation of 5 mice was used to determine an index between the spleen weight and body weight.

### Results:

A majority of the batches of LSLT prevented enlargement of the spleen after injection of the virus. In some of the batches the spleen size remained within the normal range (100-180 mg) while in others, the spleen size was significantly less than the control (300-600 mg).

### Example VII

### Effect of LSLT on Wounds in Pig Skin

### Materials and Animals:

Mini-pigs weighing about 20 kg, and anesthetics were purchased via the animal facility of the Hebrew University Medical School, Jerusalem, Israel.

Generation of wounds: The experimental protocols were approved by the ethical committee for animal research. The pigs were anesthetized by injection of 30 mg/kg kethamin, 2 mg/kg xylazin and 1 mg atropine. After shaving and disinfecting the back skin, 2 sets of 8 holes each of 2 cm diameter and to the full depth of the skin and subcutaneous fat were poked at intervals of 7-8 cm on each side of the back. The bleeding was stopped with a gauze soaked in saline with diluted adrenalin 1:1000. The initial orifice of the wounds was recorded on a translucent folio with a marker. The left side wounds were filled with 0.5 ml of either one of the two solutions A (saline control) or B (LSLT 0.5-0.6 mg protein/ml] either as a wet dressing of sterile gauze saturated with the examined compound or directly in the wound then covered with dressing material. The right side was left with only wet gauze covering the wound. The animals were then kept on analgesic treatment, until the next round of exposure to additional dose of the examined compound or the saline control.

Follow up of the wound healing: At the indicated days anesthesia, wound size plannimetry according to the crust diameter, dressing changing and repeated treatment with the treatment solution was performed. On day 14 this procedure was accompanied by biopsy of the 2 wounds from each side, treated and untreated. At the end of the experiment, after 28 days, the pigs were sacrificed by injection with an overdose of sodium pentobarbital.

Biopsy handling: The biopsy material was obtained as a punch biopsy (either 1 or 2 cm diameter). The removed trephines were fixed in 4% formic acid, embedded in paraffin blocks, cut in glass mounted sections that were prepared for H&E staining and staining for collagen by the picric acid indigo carmine procedure.

Evaluation of wound plannimetry: The recorded circles reflecting the perimeters of the wounds were evaluated by drawing the largest possible diameter and across it the largest possible crossing diameter, the wound sizes were then expressed as the mean radius size for each wound.

### Results

Tables VIII and IX show the changes in the treated wound sizes relative to the contra-lateral untreated wounds in two consecutive animals. In Table VIII the wounds were treated with solution A, while in Table IX they were treated with solution B. The first time that the relative size of treated wounds dropped sharply below 1.000 (unity) was on day 14. This was the first day after the wounds had not been treated for a long interval (last treatment was day 6) as seen for both treatment solutions A and B. It can be seen that in table IX the relative wound sizes diminished in a more consistent fashion.

TABLE VIII
Pig 54, index of wound-radius kinetics L/R (treatment A/control).

| Wound- | | | | | | | | |
|---|---|---|---|---|---|---|---|---|
| sequence | Day of wound healing | | | | | | | |
| (neck to tail) | 0 | 2 | 4 | 6 | 10 | 14 | 21 | 28 |
| 1 | 1.090 | 1.062 | 1.085 | 1.027 | 1.208 | | | |
| 2 | 0.918 | 0.916 | 0.916 | 0.972 | 1.166 | | | |
| 3 | 0.972 | 0.888 | 1.028 | 1.093 | 1.095 | 0.681 | | |
| 4 | 0.973 | 0.885 | 1.060 | 1.121 | 0.863 | 1.000 | | |
| 5 | 0.972 | 0.897 | 1.156 | 1.275 | 1.227 | 0.818 | 1.055 | |
| 6 | 0.971 | 0.941 | 1.206 | 1.241 | 1.090 | 1.043 | 0.846 | |
| 7 | 1.000 | 0.969 | 1.137 | 1.062 | 1.000 | 1.238 | 0.958 | 1.000 |
| 8 | 0.916 | 0.871 | 1.028 | 0.969 | 0.960 | 1.043 | 0.850 | 0.857 |

TABLE IX
Pig 55, index of wound-radius kinetics L/R (treatment B/control).

| Wound- | | | | | | | | |
|---|---|---|---|---|---|---|---|---|
| sequence | Day of wound healing | | | | | | | |
| (neck to tail) | 0 | 2 | 4 | 6 | 10 | 14 | 21 | 28 |
| 1 | 0.944 | 0.969 | 1.029 | 1.029 | 1.041 | | | |
| 2 | 1.000 | 0.971 | 1.030 | 1.060 | 0.958 | | | |
| 3 | 1.027 | 1.030 | 1.030 | 1.125 | 1.086 | 0.681 | | |
| 4 | 1.057 | 0.972 | 1.060 | 1.060 | 1.181 | 0.956 | | |
| 5 | 1.029 | 0.970 | 1.030 | 1.133 | 1.173 | 0.909 | 0.600 | |
| 6 | 1.057 | 1.058 | 1.000 | 1.032 | 1.040 | 0.578 | 1.000 | |
| 7 | 0.972 | 1.057 | 1.129 | 1.057 | 1.040 | 0.758 | 0.888 | 0.882 |
| 8 | 1.000 | 0.948 | 1.000 | 1.028 | 1.037 | 0.806 | 1.000 | 0.500 |

### Example VIII

### Effect of LSLT on Viral Load in Rhesus Macaques

### Methods

The study was performed in two phases:

Phase 1: Two recycled rhesus macaques (3047 and 3172) were used in this study. Both animals were previously inoculated with SHIV89.6pd on Jun. 5, 2001. An established SHIV89.6pd infection was observed in macaque 3047 as evidenced by detection of circulating virus by RT-PCR and depletion on CD4+ T-cells. No evidence of SHIV89.6pd infection was demonstrated in macaque 3172.

Dosing: Macaques 3047 and 3172 were each inoculated via multiple sites (1 ml per injection site; two sites per thigh) with 4 ml of LSLT by either subcutaneous (s/c) or intramuscular injection (i/m). Macaque 3047 received the product via the s/c route and macaque 3172 via the i/m route. Ten days later, both macaques received the same doses of the product by the same routes.

Both animals were monitored daily for any adverse reactions. Blood was collected at −7, 0, 1, 2, 3, 7, 14, 21 and 28 days for assessment of complete blood counts (CBC) by automated techniques, levels of CD4+ and CD8+ T-lymphocytes by flow cytometry and circulating IFN-γ by ELISA.

Phase 2: Six recycled SHIV162p-infected rhesus macaques, comprising two groups were used in this study. Group A consisted of three animals, namely: 3010, 3011 and 3037. On the day treatment was initiated, viral loads in these animals ranged from 2,040-16,960 RNA copies/ml plasma. Group B consisted of three animals, namely: 3012, 3013 and 3023. On the day treatment was initiated, viral loads in these animals ranged from 520-4,440 RNA copies/ml plasma.

Dosing: At 1, 2, 3 and 4 weeks, all animals in Group A were inoculated via multiple sites (1 ml per injection site; two sites per thigh) with 4 ml of LSLT by s/c injection. Animals in Group B were sham inoculated with saline, and served as controls.

All animals were monitored daily for any adverse reactions. Blood was collected weekly for assessment of complete blood counts (CBC) by automated techniques, levels of CD4+ and CD8+ T-lymphocytes by flow cytometry, viral loads and circulating IFN-γ by ELISA.

Complete Blood Counts: CBC measurements were performed by automated techniques using whole blood at LABCORP, Rockville, Md.

Flow Cytometry: CD4+ and CD8+ T-lymphocyte counts in peripheral blood were performed on a FACScalibur flow cytometer (Becton-Dickinson, Mountain View, Calif.) using phycoerythrin conjugated anti-human CD4 (CD4.PE) and peridinin chlorophyll protein conjugated anti-human CD8 (CD8.PerCP) antibodies (Becton-Dickinson). Analysis was performed using a whole blood lysis procedure as directed by the manufacturer.

Plasma Viremia: SHIV viral RNA was quantitated using a procedure described by Suryanarayana et al. (1998) AIDS Res. Hum. Retrovir. 14:183-189. Briefly, 500 μl of plasma was added to 1 ml of DPBS and spun for 1 hr at 10,000 RPM. The viral pellet was then lysed using RNASTAT-60 (Tel-Test “B”). The samples were then amplified as previously described (33), with the exception of the primers and probe. The gag primers and probe used were SIV-F 5′AGTATGGGCAGCAAATGAAT 3′, SIV-R 5′TTCTCTTCTGCGTGAATGC 3′, and the probe SIV-P 6FAMAGAT-TTGGATTAGCAGAAAGCCTGTTGGA-TAMRA. The assay has a threshold sensitivity of 200 RNA copies/ml of plasma with interassay variations averaging 0.5 log₁₀.

### Results

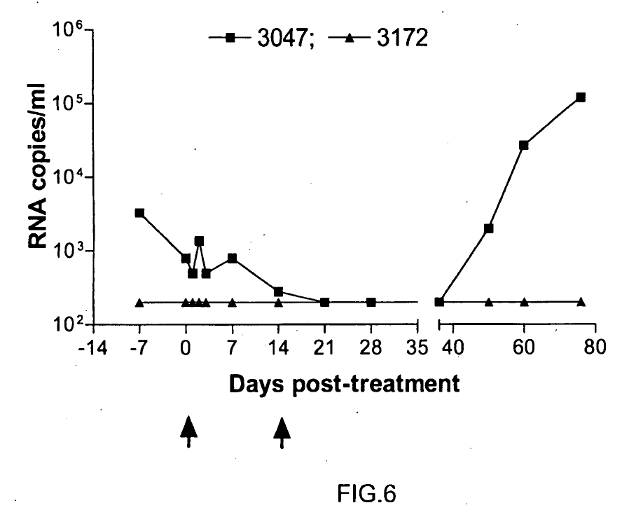

Phase 1: No adverse side-effects were observed in macaques 3047 and 3172 over the duration of the study. FIG. 6 shows the viral loads in SHIV89.6pd-infected macaques 3047 and 3172, before, during and after LSLT treatment. Although 3172 was inoculated with SHIV89.6pd, no virus infection was detected throughout the study (FIG. 6). This was evidenced by maintenance of CD4+ T-lymphocyte levels.

An established SHIV89.6pd infection was clearly demonstrated in macaque 3047 (FIG. 6) as evidenced by depletion of CD4+ T-lymphocytes that remained below 10% throughout the duration of the study. Seven days prior to initiation of LSLT treatment, 3.3×10³ SHIV89.6pd RNA copies/ml were detected in macaque 3047, and on the day of the first LSLT injection, the viral load in this animal was 800 RNA copies/ml (FIG. 6). Seven days after the second injection (day 21), SHIV viral RNA was undetectable in macaque 3047 and remained so through to day 36. However, viral loads rebounded at day 50, and continued to rise thereafter.

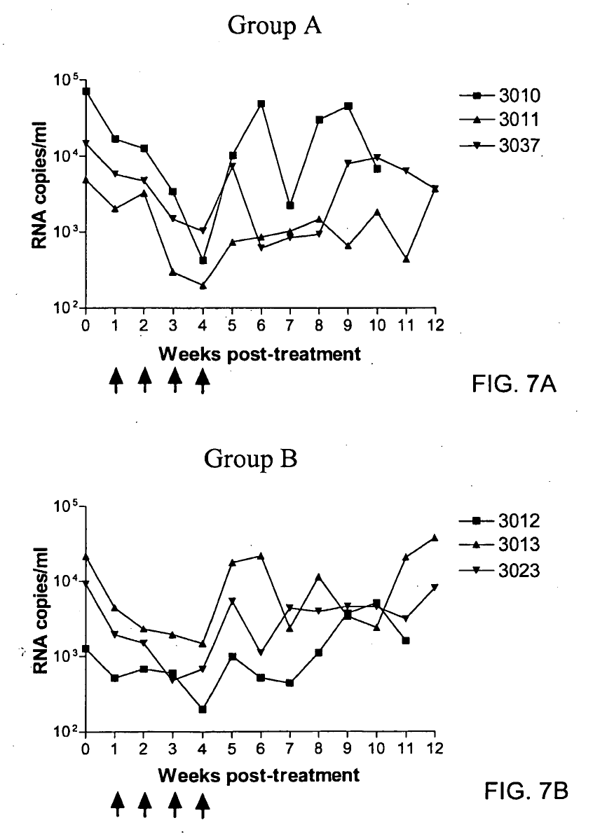

Phase 2: Illustrated in FIG. 7 are viral load measurements for Group A and B during and following treatment with either the LSLT or saline. On the day of initiation of treatment with LSLT (week 1), viral loads in Group A macaques 3010, 3011 and 3037 ranged from 2,040-16,960. After three doses of LSLT, viral loads in these animals decreased to values ranging from <200-1,040 RNA copies/ml by week 4, representing a 10 to 16-fold reduction in circulating virus. However, after the final dose given at week 4, viral loads rebounded in all three animals to varying degrees. Animal 3010 died soon after week 10. This animal had the lowest CD4+ T-lymphocyte count for the duration of the study. Animals in Group B (3012, 3013 and 3023) given saline only, had viral loads ranging from 520-4440 RNA copies/ml on week 1. These levels had decreased to values ranging from <200-1,480 RNA copies on week 4, representing a 2.6 to 3-fold reduction. However, all animals experienced a rebound in their viral loads. Such fluctuating patterns of viremia have been observed in other studies involving SHIV162p infection of rhesus macaques.

### Example IX

### Evaluation of the Activity of LSLT (Crude Material) and its Fractions

The goal of this experiment was to test the potency of LSLT crude material (the bacterial supernatant concentrated 10×) and fractions thereof obtained by filtering as in Example III above.

### Methodology

The potency of the LSLT or its fractions is based on the appearance of splenomegaly in mice inoculated with the Rauscher-Like Murine Leukemia Virus (RL-MuLU), 2-3 weeks post inoculation (see Example VI above).

The infected spleen has a size 3-6 times greater than in uninfected mice. Simultaneous administration of LSLT with the virus, inhibits splenomegaly either totally or to a significant extent.

### Material and Methods

Mice: Balb C: 8 weeks old. (Harlan Inc.)

Virus: batch 7-0.2 ml of virus diluted 1:10 in plasma were inoculated into each mouse. The number of mice in each set are indicated.

LSLT and Fractions:

1) LSLT batch 41 which contains 0.850 mg/ml protein (10 mice)

2) LSLT batch 41 treated 96° C. for 30′. (9 mice)

3) Filtered fraction 12 KD and above (10 mice)

4) Filtered fraction 7-12 KD (12 mice)

5) Filtered fraction 3.5-7 KD (15 mice)

6) Filtered fraction 3-3.5 KD (8 mice)

7) Control mice with no treatment (6 mice)

Each mouse received 0.5 ml substance, 20 min prior to viral inoculation

Fractionation of the LSLT was obtained by a filtering process based on molecular weight.

Evaluation: all mice were sacrificed 20 days post inoculation.

The Body Weight (BW) and spleen weight (SW) of each mouse were measured and a coefficient was determined which was calculated by the ratio of BW/SW for each group. Thus, the percentage of the reduction of splenomegaly could be evaluated.

### Results

The summary of the results obtained is given in Table X.

TABLE X
The potency of LSLT and its fractions as evaluated
by the reduction of splenomegaly

| Group | Type of material | No. of mice | % of reduction |
|---|---|---|---|
| 1 | Virus only | 10 | 0 |
| 2 | Virus + SLST | 10 | 30 |
| 3 | Virus + heated LSLT | 9 | 60 |
| 4 | Virus + 12 KD> | 10 | 20 |
| 5 | Virus + 7 - 12 KD | 12 | 30 |
| 6 | Virus + 3.5 - 7 KD | 15 | 60 |
| 7 | Virus + 3 - 3.5 KD | 8 | 60 |
| 8 | no treatment | 6 | 100 |

A significant reduction (60%) could be observed in groups 3, 6 and 7, namely the heated crude material and the fractions 3.5-7 KD and 3-3.5 KD. These results indicate that the active substance is heat resistant and has a MW less than 3 KD.

None of the materials tested here gave a total reduction (100%). It is assumed that it could have resulted from the fractionation procedure; namely the materials were diluted and were not at their original concentration.

1. A substance obtainable from Staphylococcus Aureus bacteria or from a Staphylococcus Aureus bacteria culture, the substance characterized in having at least one of the following characteristics:
(a) stimulates proliferation of bovine T-lymphocytes in vitro;
(b) accelerates healing of skin wounds in mice and pigs;
(c) prevents AIDS-like disease (ALD) in mice infected with ALD virus (ALD-V) when injected substantially simultaneously with the virus; and
(d) reduces the viral load in Rhesus Macaques infected with the hybrid simian/human immunodeficiency virus (SHIV) when injected into chronically infected Macaques, or a derivative or fraction of said substance which retains at least one of the characteristics of the substance.

2. The substance of claim 1 having at least two of characteristics (a), (b), (c) and (d).

3. The substance of claim 1 having at least three of characteristics (a), (b), (c) and (d).

4. The substance of claim 1 having all of characteristics (a), (b), (c) and (d).

5. The substance of claim 1 being the supernatant of a Staphylococcus Aureus bacteria culture or being a molecular entity obtainable therefrom.

6. The substance of claim 1 which retains at least characteristic (c) after being heated to 96° C. for 30 minutes.

7. A derivative, homologue or analog of the molecular entity of claim 5 which retains at least characteristics (c) and (d) of said substance.

8. A Staphylococcus Aureus bacterium deposited at the National Collection of Agricultural and Industrial Microorganisms, Budapest, Hungary, on Jul. 6, 2004 under the accession number NCAIM (P) B 001321.

9. The substance according to claim 1 wherein the Staphylococcus Aureus bacterium is that deposited at the National Collection of Agricultural and Industrial Microorganisms, Budapest, Hungary, on Jul. 6, 2004 under the accession number NCAIM (P) B 001321.

10. A pharmaceutical composition comprising as active ingredient the substance of claim 1 and a pharmaceutically acceptable excipient.

11. (canceled)

12. The pharmaceutical composition of claim 10 for the treatment of AIDS.

13. A pharmaceutical composition comprising as active ingredient the derivative, homologue or analog of claim 7 and a pharmaceutically acceptable excipient.

14. (canceled)

15. (canceled)

16. A method for healing a wound of a subject comprising administrating to the subject the substance of claim 1.

17. The method of claim 16 wherein the substance is administered topically.

18. The method of claim 16 wherein the substance is administered subcutaneously (sc).

19. A method for the prevention or treatment of infection of a subject by the human immunodeficiency virus (HIV) or by animal retroviruses, or for the treatment of a consequent pathological condition thereof, comprising administrating to the subject the substance of claim 1.

20. The method of claim 19 wherein said consequent pathological condition is AIDS.

21. Use of the substance of claim 1 in the preparation of a pharmaceutical composition.

22. (canceled)

23. (canceled)

24. A method for preparing a substance according to claim 5, the method comprising:
(a) growing a Staphylococcus Aureus bacteria culture in a bacterial growth medium from a bacteria species equivalent to that deposited at the National Collection of Agricultural and Industrial Microorganisms, Budapest, Hungary, on Jul. 6, 2004 under the accession number NCAIM (P) B 001321; and
(b) separating the bacteria and concentrating the supernatant to obtain said substance.

25. The method of claim 24 further comprising the steps of:
(c) applying the substance of step (b) to an RP-18 column; and
(d) eluting an active fraction from the column, said fraction having at least one of the characteristics (a), (b), (c) and (d) of the substance of claim 1.
